(19) United States
(12) Patent Application Publication
(10) Pub. No.: US 2008/0281390 A1
(43) Pub. Date: Nov. 13, 2008

(54) MAGNETOSTRICTIVE ELECTRICAL STIMULATION LEADS

(76) Inventor: MARK T. MARSHALL, Forest Lake, MN (US)

Correspondence Address: MEDTRONIC, INC., 710 MEDTRONIC PARKWAY NE, MINNEAPOLIS, MN, 55432-9924, US

(21) Appl. No.: 12/110,806

(22) Filed: Apr. 28, 2008

Related U.S. Application Data
(63) Continuation-in-part of application No. 11/741,601, filed on Apr. 27, 2007.

(51) Int. Cl. A61N 1/05

(52) U.S. Cl. 607/116

(57) ABSTRACT

A medical device lead is presented. The medical device lead includes a lead body, an electrode shaft, and a tip electrode. A magnetostrictive element is coupled to the electrode shaft. The magnetostrictive element comprises either terfenol-D and/or galfenol or any material with sufficient magnetostrictive properties. The magnetostrictive element expands when exposed to magnetic resonance imaging.

### CROSS-REFERENCE TO RELATED APPLICATION

This application is a continuation-in-part of U.S. patent application Ser. No. 11/741,601 (Attorney docket P27828.00) filed on filed Apr. 27, 2007 and entitled “MAGNETOSTRICTIVE ELECTRICAL STIMULATION LEADS”, the disclosure of the above-referenced application is incorporated herein by reference. The present disclosure is also related to another application entitled MAGNETOSTRICTIVE ELECTRICAL STIMULATION LEADS, U.S. Application Ser. No. 11/741,602, filed Apr. 27, 2007.

### TECHNICAL FIELD

The present disclosure relates to medical devices and, more particularly, to implantable medical device leads for use with implantable medical devices (IMDs).

### BACKGROUND

In the medical field, implantable leads are used with a wide variety of medical devices. For example, implantable leads are commonly used to form part of implantable cardiac pacemakers that provide therapeutic stimulation to the heart by delivering pacing, cardioversion or defibrillation pulses. The pulses can be delivered to the heart via electrodes disposed on the leads, e.g., typically near distal ends of the leads. In that case, the leads may position the electrodes with respect to various cardiac locations so that the pacemaker can deliver pulses to the appropriate locations. Leads are also used for sensing purposes, or for both sensing and stimulation purposes. Implantable leads are also used in neurological devices, muscular stimulation therapy, and devices that sense chemical conditions in a patient's blood, gastric system stimulators.

Occasionally, patients that have implantable leads may benefit from a magnet resonance image being taken of a particular area of his or her body. Magnetic resonance imaging (MRI) techniques achieve a more effective image of the soft tissues of the heart and vascular system. MRI procedures can also image these features without delivering a high dosage of radiation to the body of the patient, and as a result, MRI procedures may be repeated reliably and safely. However, MRI devices may operate at frequencies of 10 megahertz or higher, which may cause energy to be transferred to the lead. It is desirable to develop new MRI-safe leads.

### BRIEF DESCRIPTION OF DRAWINGS

Aspects and features of the present invention will be appreciated as the same becomes better understood by reference to the following detailed description of the embodiments of the invention when considered in connection with the accompanying drawings, wherein:

FIG. 1 is a schematic view of an implantable medical device placed in a human body;

FIG. 2 is a schematic view of another implantable medical device placed in a human body.

FIG. 3 is a conceptual perspective view of a medical device system including a medical device coupled to a lead according to an embodiment of the present invention;

FIG. 4 is a cross-sectional view of an electrode assembly located at a distal end of a medical lead;

FIG. 5 depicts multiple layers of insulating material over a conductive element of the electrode assembly depicted in FIG. 4;

FIG. 6A depicts a cross-sectional view of a conductive ring coupled to a conductive sealer for the electrode assembly depicted in FIG. 4;

FIG. 6B depicts a top view of a conductive ring coupled to a conductive sealer for the electrode assembly depicted in FIG. 4;

FIG. 6C depicts an exploded view of conductive rings and a conductive sealer coupled to a shaft;

FIG. 6D depicts an angled view of a conductive sealer;

FIG. 7A is a schematic diagram of a simplified bipolar circuit for a medical device system under pacing and sensing conditions;

FIG. 7B is a schematic unipolar circuit for a medical device system;

FIG. 8A is a schematic bipolar circuit for a simplified medical device system;

FIG. 8B is a schematic bipolar circuit of another simplified circuit for a medical device system;

FIG. 9A depicts a cross-sectional view of an electrode assembly with a magnetostrictive element disposed between a first and a second segment of an electrode shaft in which the first segment directly contacts the second segment;

FIG. 9B depicts a cross-sectional view of an electrode assembly with an expanded magnetostrictive element that separates the first segment from the second segment of an electrode shaft; and

FIG. 10 is a flow diagram that depicts a method of producing an electrode assembly.

### DETAILED DESCRIPTION

The present disclosure is directed to a medical lead, techniques for manufacturing such a lead, and systems that include a medical device coupled to a medical lead according to the present invention. The medical device lead includes a lead body, an electrode shaft and a tip electrode. A magnetostrictive element, coupled to an electrode shaft, serves as an “on/off” switch to manage high frequency RF signals (e.g. 21 megaHertz (Mhz) to 128 MHz) generated from a magnetic resonance imaging (MRI) machine away from the tip electrode. The switch is comprised of a magnetostrictive element made of any suitable material with sufficient magnetostrictive properties. Exemplary magnetostrictive material includes terfenol-D or galfenol. Magnetostriction is a property that causes certain ferromagnetic materials to change shape in response to a magnetic field. In particular, the magnetostrictive element expands or contracts. When the lead is not exposed to a MRI, the magnetostrictive material is contracted. In contrast, when the lead is exposed to MRI, the magnetostrictive material expands. In one embodiment, expansion of the magnetostrictive material causes a first segment to move away from a second segment of the electrode shaft. A gap is created between the first and second segments of the electrode shaft. Therefore, current, induced in the lead due to exposure to the MRI, no longer has a direct electrical path to the tip electrode. Instead, the electrical current induced by high frequency passes through a high impedance component such as a radiofrequency (RF) trap, whereas the low frequency current for sensing and/or pacing is able to pass to and/or from the electrode tip. Consequently, a patient with a medical lead may undergo an MRI procedure without significantly affecting the operation of the medical lead.

In another embodiment, magnetostrictive material is disposed in or near conductive members (e.g. C-rings) that are coupled to the electrode shaft. When the lead is exposed to MRI, the magnetostrictive material expands to create a contact to an additional electrode surface, which allows the induced current to dissipate over a larger surface area. In one embodiment, a tenfold (i.e. 10×) larger surface area ratio results in about tenfold lower temperatures at the tip electrode assuming a ring electrode has low impedance at high frequencies. The principles described herein are applicable to all types of medical electrical leads. For example, the disclosure applies to cardiovascular leads (e.g. high voltage leads, low voltage leads etc.), neurological leads, or other suitable applications. Any size of lead can be used such as a size 13 French lead or less.

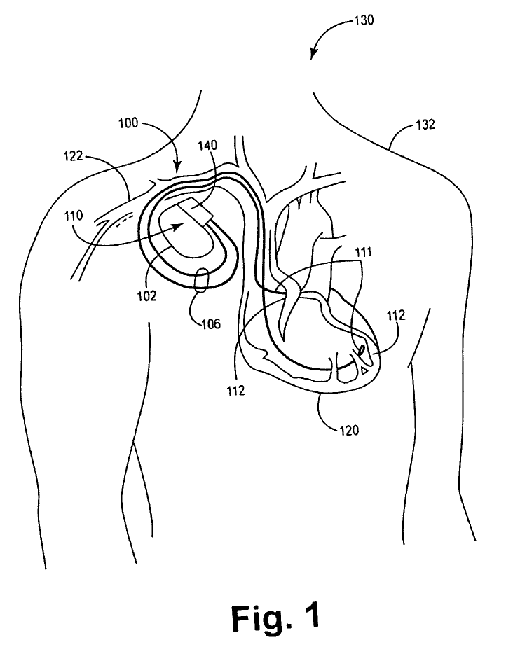

FIG. 1 illustrates a schematic view of a medical device system 100 (e.g., an implantable medical device or IMD) implanted within a body or torso 132 of a patient 130. The medical device system 100 may include electrodes having thermally and/or magnetostrictive sensitive switches. The medical device system 100 includes a device 110 in the form of an implantable medical device that for purposes of illustration is shown as a defibrillator configured to provide a therapeutic high voltage (e.g., 700 volt) treatment for the patient 130.

The device 110 includes a container or housing 102 that is hermetically sealed and biologically inert according to an exemplary embodiment. The container may be made of a conductive material. One or more leads 106 electrically connect the device 110 to the patient's heart 120 via a vein 122. Electrodes are provided to sense cardiac activity and/or provide an electrical potential to the heart 120. At least a portion of the leads 106 (e.g., an end portion of the leads shown as exposed electrodes 111 attached to heart tissue 112) may be provided adjacent or in contact with a ventricle and/or an atrium of the heart 120.

The device 110 includes a battery 140 provided therein to provide power for the device 110. The size and capacity of the battery 140 may be chosen based on a number of factors, including the amount of charge required for a given patient's physical or medical characteristics, the size or configuration of the device, and any of a variety of other factors. According to an exemplary embodiment, the battery is a 5 milliampere hour (mAh) battery. According to another exemplary embodiment, the battery is a 300 mAh battery. According to various other exemplary embodiments, the battery may have a capacity of between approximately 10 and 1000 mAh.

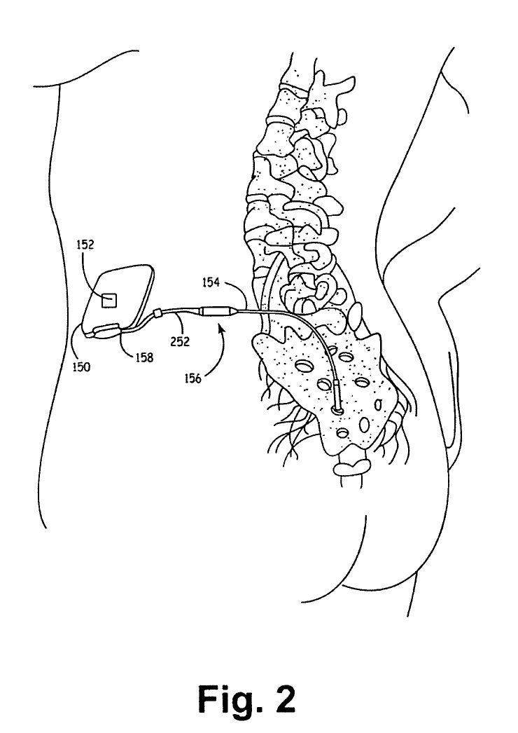

According to another exemplary embodiment shown in FIG. 2, an implantable neurological stimulation device 150 (an implantable neurostimulator or INS) may include a battery 152 such as those described above with respect to the various exemplary embodiments. Examples of some neurostimulation products and related components are shown and described in a brochure titled “Implantable Neurostimulation Systems” available from Medtronic, Inc.

An INS generates one or more electrical stimulation signals that are used to influence the human nervous system or organs. Electrical contacts carried on the distal end of a lead are placed at the desired stimulation site such as the spine or brain and the proximal end of the lead is connected to the INS. The INS is then surgically implanted into an individual such as into a subcutaneous pocket in the abdomen, pectoral region, or upper buttocks area. A clinician programs the INS with a therapy using a programmer. The therapy configures parameters of the stimulation signal for the specific patient's therapy. An INS can be used to treat conditions such as pain, incontinence, movement disorders such as epilepsy and Parkinson's disease, and sleep apnea. Additional therapies appear promising to treat a variety of physiological, psychological, and emotional conditions.

The INS 150 typically includes a lead extension 152 and a stimulation lead 154. The stimulation lead 154 is one or more insulated electrical conductors with a connector 156 on the proximal end and electrical contacts (not shown) on the distal end. Some stimulation leads are designed to be inserted into a patient percutaneously, such as the Model 3487A Pisces-Quad® lead available from Medtronic, Inc. of Minneapolis Minn., and some stimulation leads are designed to be surgically implanted, such as the Model 3998 Specify® lead also available from Medtronic. Although the lead connector 156 can be connected directly to the INS 150 (e.g., at a point 158), typically the lead connector 156 is connected to a lead extension 152. The lead extension 152, such as a Model 7495 available from Medtronic, is then connected to the INS 150. Implantation of an INS 150 typically begins with implantation of at least one stimulation lead 154, usually while the patient is under a local anesthetic. The stimulation lead 154 can either be percutaneously or surgically implanted. Once the stimulation lead 154 has been implanted and positioned, the stimulation lead's 154 distal end is typically anchored into position to minimize movement of the stimulation lead 154 after implantation. The stimulation lead's 154 proximal end can be configured to connect to a lead extension 152. The INS 150 is programmed with a therapy and the therapy is often modified to optimize the therapy for the patient (i.e., the INS may be programmed with a plurality of programs or therapies such that an appropriate therapy may be administered in a given situation).

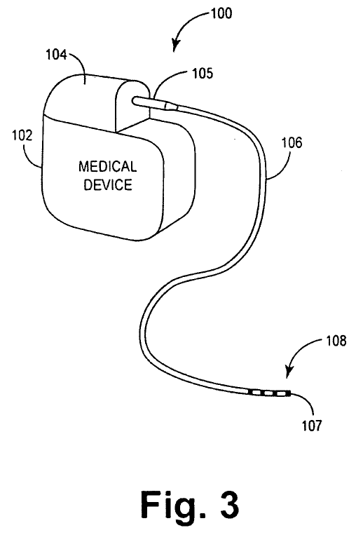

FIG. 3 depicts a medical device system 100. A medical device system 100 includes a medical device housing 102 having a connector module 104 (e.g. international standard (IS)-1, defibrillation (DF)-1, IS-4 etc.) that electrically couples various internal electrical components housed in medical device housing 102 to a proximal end 105 of a medical lead 106. A medical device system 100 may comprise any of a wide variety of medical devices that include one or more medical lead(s) 106 and circuitry coupled to the medical lead(s) 106. An exemplary medical device system 100 can take the form of an implantable cardiac pacemaker, an implantable cardioverter, an implantable defibrillator, an implantable cardiac pacemaker-cardioverter-defibrillator (PCD), a neurostimulator, a tissue and/or muscle stimulator. IMDs are implanted in a patient in an appropriate location. Exemplary IMDs are commercially available as including one generally known to those skilled in the art, such as the Medtronic CONCERTO®, SENSIA®, VIRTUOSO®, RESTORE®, RESTORE ULTRA®, sold by Medtronic, Inc. of Minnesota. Non-implantable medical devices or other types of devices may also utilize batteries such as external drug pumps, hearing aids and patient monitoring devices or other suitable devices. Medical device system 100 may deliver, for example, pacing, cardioversion or defibrillation pulses to a patient via electrodes 108 disposed on distal end 107 of one or more lead(s) 106. Specifically, lead 106 may position one or more electrodes 108 with respect to various cardiac locations so that medical device system 100 can deliver electrical stimuli to the appropriate locations.

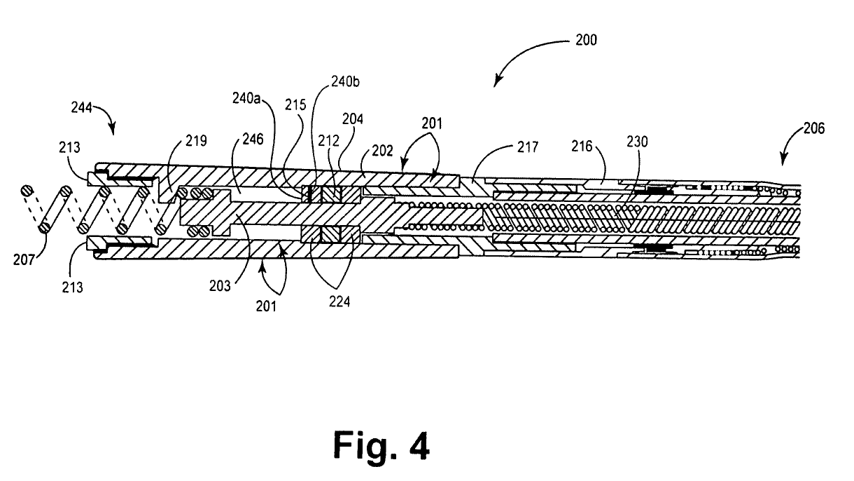

FIG. 4 depicts an electrode assembly 200 of a medical lead 106. Electrode assembly 200 senses physiologic data from a patient and/or delivers electrical stimuli to tissue of a patient. Electrode assembly 200 optionally includes a sleeve head 201 coupled to an electrode 207 (also referred to as a tip electrode), a monolithic controlled-release device (MCRD) 213, a conductive electrode shaft 203, a conductive sealer 212, conductive members 224, a ring electrode 216, and a non-conductive spacer 217. At a distal end 244 of electrode assembly 200, a sharpened distal tip (not shown) facilitates fixation of the distal end of helically shaped electrode 207 into tissue of a patient. The proximal end of electrode 207 is securely seated between MCRD 213, electrode shaft 203, and a securing member 219 that protrudes from an inner diameter of sleeve head 201. MCRD 213 provides chronic steroid elution to maintain a low pacing threshold for a medical device system 100.

Sleeve head 201 (optionally, a RF-shunted sleeve head) is electrically connected to a conductive electrode shaft 203 (e.g. platinum etc.) via two parallel conductive members 224 (e.g. C-rings, etc.) a conductive sealer 212 (also referred to as a sealing washer), and a magnetostrictive element 215 insulated with insulative layer 260. Insulative layer 260 is comprised of, for example, hydrolytically stable polyimide (commonly referred to as Langley Research Center SI (“LaRC SI”) commercially available from Imitec located in Schenectady, N.Y.) Other insulative materials can also be used such as fluoropolymers (polytetrafluroethylene (PTFE), tetrafluroethylene (ETFE), etc.), thermoplastics (polyether ether ketone (PEEK), polyethersulfone (PES), etc.) and ceramic oxides (alumina, tantalum oxide, etc.). At a proximal end 206 of electrode assembly 200, coil 230 is electrically coupled to conductive electrode shaft 203. In another embodiment, electrode shaft 203 is made of nonconductive polymeric material.

Sleeve head 201 comprises a conductive element 202 surrounded or at least partially covered by an insulating material 204 (also referred to as a dielectric material). In one embodiment, conductive element 202 is cylindrically shaped (e.g. ring, etc.) or may possess other suitable shapes. Exemplary dimensions for conductive element 202 include a diameter of about 6.5 French (Fr.) by about 9 millimeters (mm) in length, an outer diameter of about 82 mils and an inner diameter of about 62 mils. Conductive element 202, in one embodiment, includes an increased diameter at the distal end and a reduced diameter at the proximal end of the conductive element 202. The surface area of conductive element 202 is about 60 mm² which is much larger than the 5.5 mm² surface area of electrode 207. Conductive element 202 comprises materials that are chemically stable, biocompatible, and x-ray transparent. Exemplary material used to form conductive element 202 includes titanium, titanium alloy, conductive polymers, and/or other suitable materials.

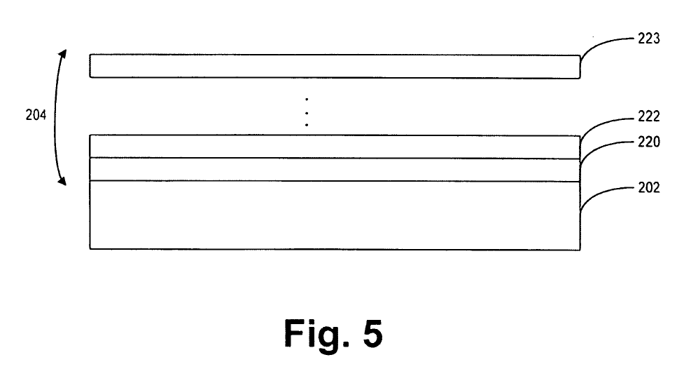

Referring to FIG. 5, insulative material 204 may be formed from a single layer or multiple layers such as first layer 220, second layer 222, and N layer 223, where N is a whole number that is less than 100, and is typically less than about 30 layers. Each layer may comprise different insulating materials, two or more different insulating materials, or the same insulating materials. Insulative material 204 includes a thickness from about 1 nanometer (nm) to about 1 millimeter (mm)) and extends from about 1 mm to about 20 mm along the length of conductive element 202. Insulative material 204 may be formed from any of a wide variety of insulating materials. Exemplary insulating material comprise at least one or more of parylene, polyamide, metal oxides, polyimide, urethane, silicone, ETFE, PTFE, expanded PTFE (i.e. porous and/or nonporous ePTFE), or the like. Parylene is the preferred insulating material 204. The preferred parylene is parylene C. Parylene C is formed through a dimer vacuum deposition process. The dimer is commercially available from Specialty Coating Systems located in Clear Lake, Wis. Numerous techniques may be employed to introduce insulating material 204 over the outside of sleeve head 201 and/or partially inside sleeve head 201. Exemplary techniques include chemical vapor deposition, dip coating, or thermal extrusion.

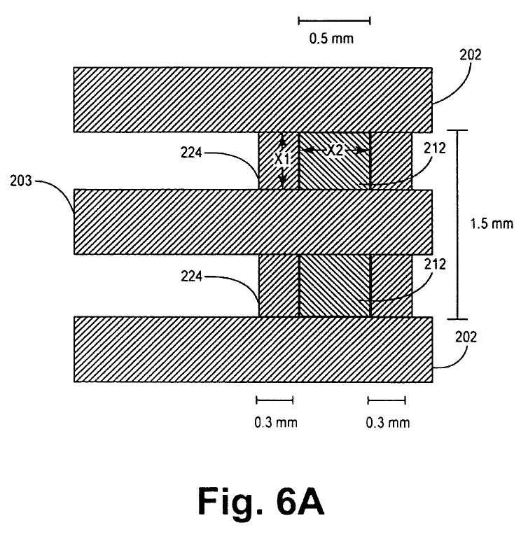

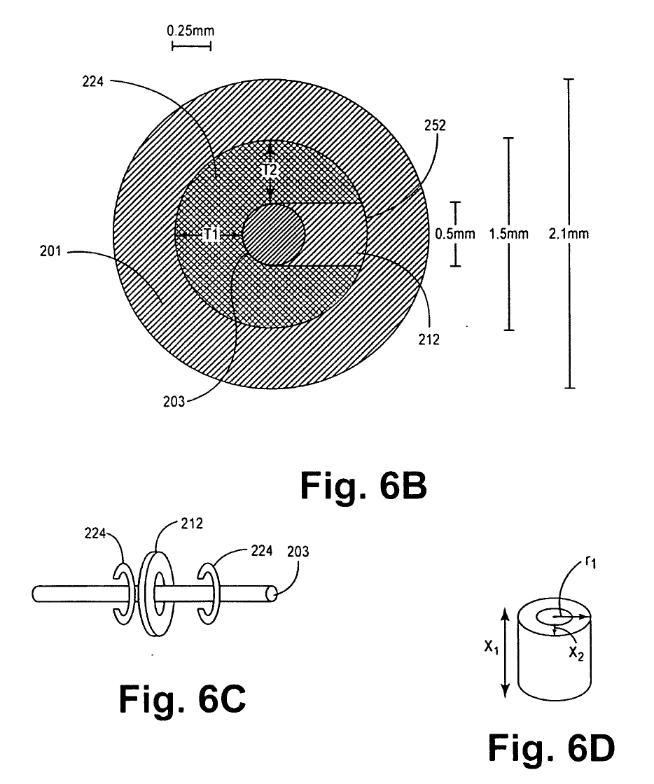

Conductive sealer 212 conducts current and also prevents fluid from passing through lumen 246. Referring to FIGS. 6A-6D, conductive sealer 212 is substantially ring (i.e. o-ring) or disk shaped but other suitable shapes may also be employed. In one embodiment, conductive sealer 212 is defined by X1, X2 and radius (r1). X1 ranges from about 0.1 mm to about 0.50 mm, X2 extends from about 0.1 mm to about 1.0 mm, and r1 extends from about 0.5 mm to about 1.0 mm. Curved end 252 extends to about 1.25 mm from the center of shaft 203 and includes a curve defined by a radius of about 0.5 mm.

Conductive sealer 212 comprises a polymer and a conductive polymer such as a conductive powder (e.g. carbon, carbon nanotube, silver, platinum etc.). The conductive polymer ranges from about 1% to about 25% of conductive sealer 212. The polymer (e.g. silicone etc.) is commercially available from Nusil Technology LLC, located in Carpinteria, Calif. Polyurethane is commercially available from The Polymer Technology Group Inc. located in Berkeley, Calif.

Conductive members 224 are shaped, in one embodiment, as a ring (e.g. C-ring etc.) to receive conductive sealer 212. Conductive members 224 have an outer diameter of about 1.5 mm, an inner diameter of about 0.7 mm, and a thickness that ranges from about 0.25 mm (T1) to about 0.5 mm (T2). Conductive members 224 are comprised of platinum or other suitable materials.

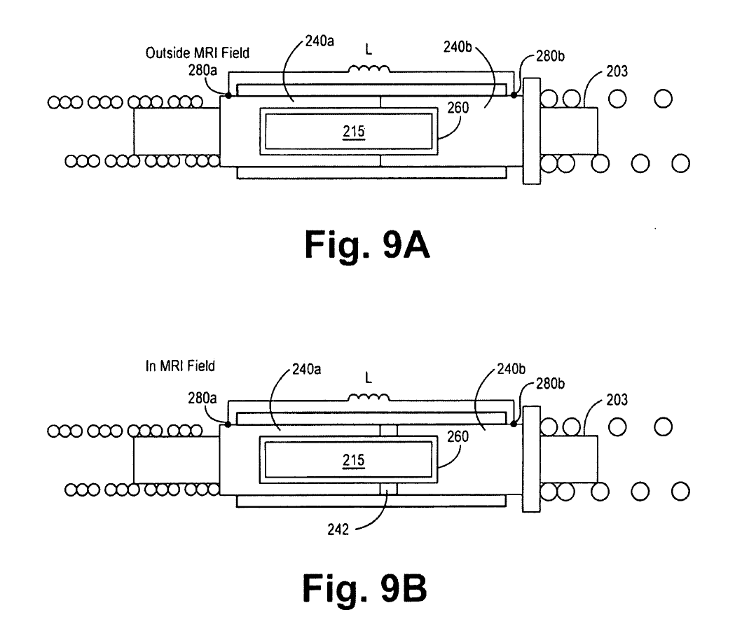

In one embodiment, magnetostrictive element 215 is coupled to at least one conductive member 224. When lead 106 is exposed to MRI, magnetostrictive element 215 expands, which creates a larger surface area in which to dissipate the current induced in lead 106. In another embodiment, depicted in FIGS. 9A-9B, magnetostrictive element 215, is disposed between first and second segments 240a,b of electrode shaft 203. The magnetostrictive element 215 may be coated with an insulative material layer (such as polyimide) in a cylindrical shape of approximately 0.5 mm-2.0 mm in diameter by 1.0 mm-4.0 mm long and bonded at the ends of segment 240 a,b. The outer surface of members 240a, b may also maintain contact through an elastic member assembled on the outside diameter. No gap exists between first and second segments 240a,b when MRI is not applied to lead 106. When lead 106 is exposed to MRI, magnetostrictive element 215 expands, causing first segment 240a to move away from second segment 240b, thereby creating a gap 242. Gap 242 breaks the direct electrical connection between first and second segments 240a,b and the tip electrode 207. Instead, the current induced by MRI is shunted to a RF trap. In particular, high impedance inductor (L) 262, connected to electrode shaft 203, blocks the high frequency RF signals. L passes the low frequency pacing signals from one end 280a to another end 280b of the electrode shaft 203. The high frequency RF signals are shunted to magnetostrictive element 215, as shown in FIG. 9B.

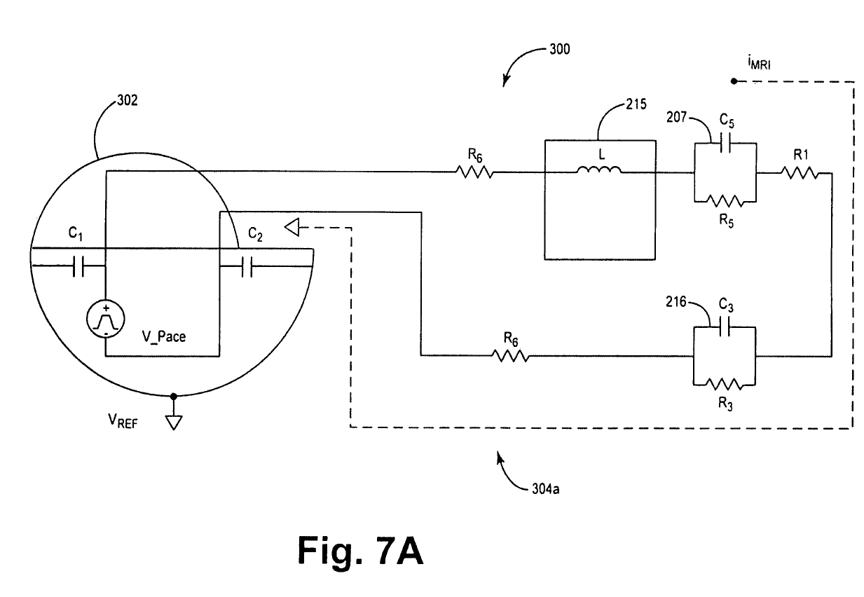

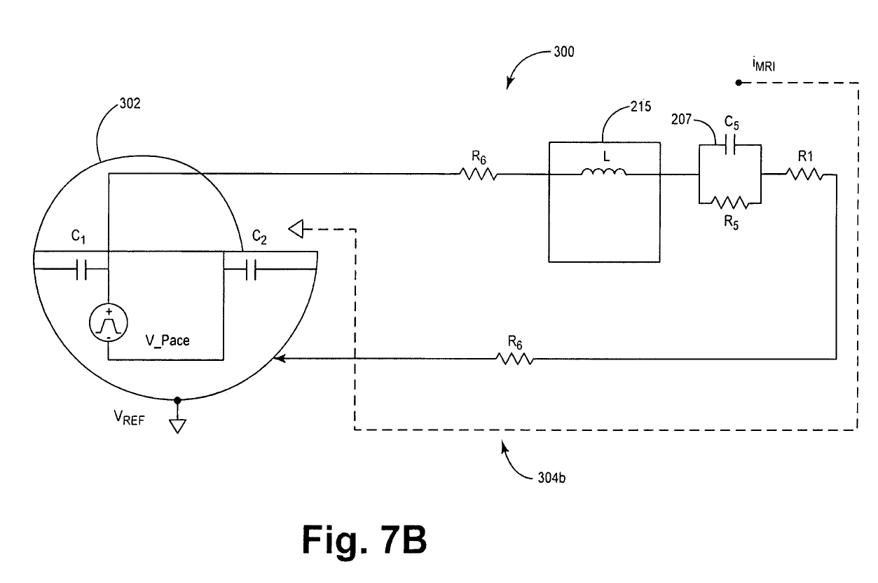

FIG. 7A depicts a simplified bipolar circuit 300 for a medical device system 100 during normal pacing conditions and when exposed to MRI. Pacing conditions typically involve low frequency signals (e.g. 1000 Hz). Circuit 300 includes an implantable medical device (IMD) circuit 302 (e.g. a pacemaker circuit, neurostimilator circuit etc.) connected to a bipolar shunted lead circuit 304. IMD circuit 302 comprises two filter capacitors C1 and C2 connected to housing 102. C1 and C2 filter high frequency electromagnetic interference (EMI) so that high frequency signals from a MRI machine do not affect the sensing operation of medical lead 106. Exemplary values for C1 is about 1 to 10 nanoFarad (nF) and C2 is 1-10 nF.

Bipolar shunted lead circuit 304a includes ring electrode 216, magnetostrictive element 215, and tip electrode 207. Capacitors C3 and C5 correspond to ring electrode 216, and tip electrode 207, respectively and inductor L is associated with magnetostrictive element 215. Resistors R1 represents the impedance created by tissue and/or blood of the patient. R6 represents impedance or resistance associated with coil 230. R3 and R5, along with capacitors C3 and C5, represent the electrode to tissue interface impedances. V_{REF} represents the device ground to the body whereas V_{—}_{^{Pace}} represents the voltage of the pacing current. Generally, larger area electrodes result in larger values of capacitance and smaller values of resistance. Exemplary values for bipolar shunted lead circuit 304a include C3 at 10 microF (uF), L is 4 uHenry, R3 is 100 Ohm (Ω), C5 is 1 uF, and R1 is 500Ω, and R5 is Ω.

Bipolar shunted lead circuit 304a operates when the patient is not exposed to a MRI procedure or during a MRI procedure. When MRI conditions are not present and pacing pulses are required by a patient, pacing current is generated from C1 or C2 and passes through first and second segments 240a, 240b, as shown in greater detail in FIG. 9A. Thereafter, the pacing current passes through tip electrode 207, ring electrode 216 and back to medical device housing 102.

When a patient is exposed to a MRI procedure, a MRI current (I_{MRI}) is transferred from tip electrode 207 through ring electrode 216 back to medical device housing 102, which avoids or substantially prevents the MRI from affecting the operation of lead 106 from delivering electrical stimuli (i.e. pacing pulses) to the patient. If the patient requires pacing during the MRI, the pacing current, typically generated from C1, passes through an inductor (L), depicted in FIG. 9B, and thereafter passes through tip electrode 207, ring electrode 216 and then back to medical device housing 102.

FIG. 7B depicts a simplified unipolar circuit 300 for a medical device system 100 during normal pacing conditions and when exposed to MRI. Circuit 300 includes an IMD circuit 302 (e.g. a pacemaker circuit, neurostimilator circuit etc.) connected to a unipolar shunted lead circuit 304b. IMD circuit 302 comprises two filter capacitors C1 and C2 connected to housing 102. C1 and C2 filter high frequency EMI so that high frequency signals from a MRI machine do not affect the sensing operation of medical lead 106.

Unipolar shunted lead circuit 304b includes magnetostrictive element 215, and tip electrode 207. Capacitor C5 corresponds to tip electrode 207 and inductor L is associated with magnetostrictive element 215. Resistor R1 represents the impedance created by tissue and/or blood of the patient. R6 represents impedance or resistance associated with coil 230. R5 along with capacitor C5, represent the electrode to tissue interface impedance.

Generally, under typical pacing conditions, pacing current flows from C1 or C2 through tip electrode 207 and then returns to IMD circuit 302. Under a low frequency or DC application, inductor L acts like a short circuit to a constant voltage across its terminals. A portion of the pacing current passes to the patient's tissue (e.g. heart tissue), represented as resistor R1, due to the large capacitance of C5 associated with tip electrode 207. When lead 106 is exposed to MRI, I_{MRI} is induced, as depicted by the ghost lines. As shown, the I_{MRI} is transferred from tip electrode 207 back to medical device housing 102. If the patient requires pacing during an MRI procedure, a pacing current is generated from C1 or C2 and passes through inductor L, (shown in FIG. 9B), tip electrode 207 back to medical device housing 102.

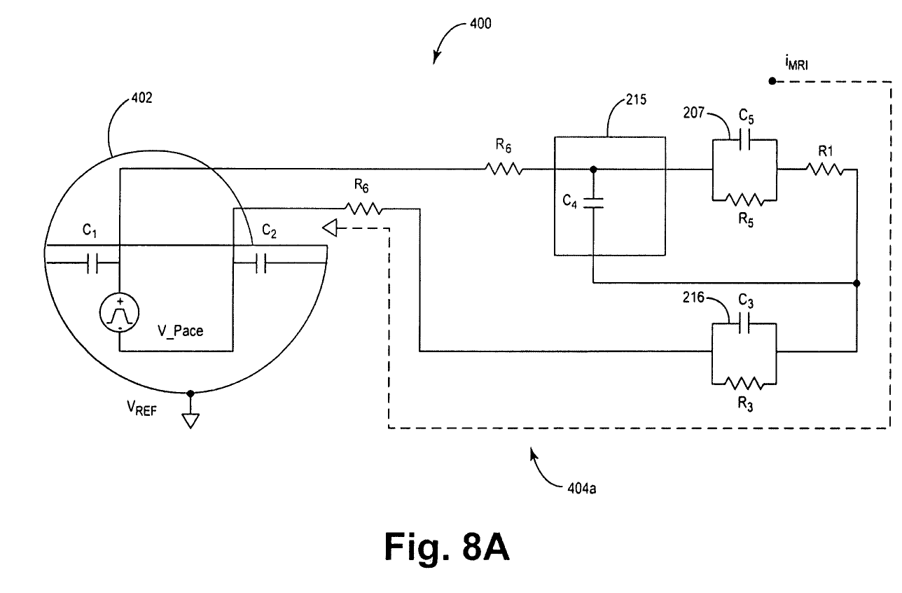

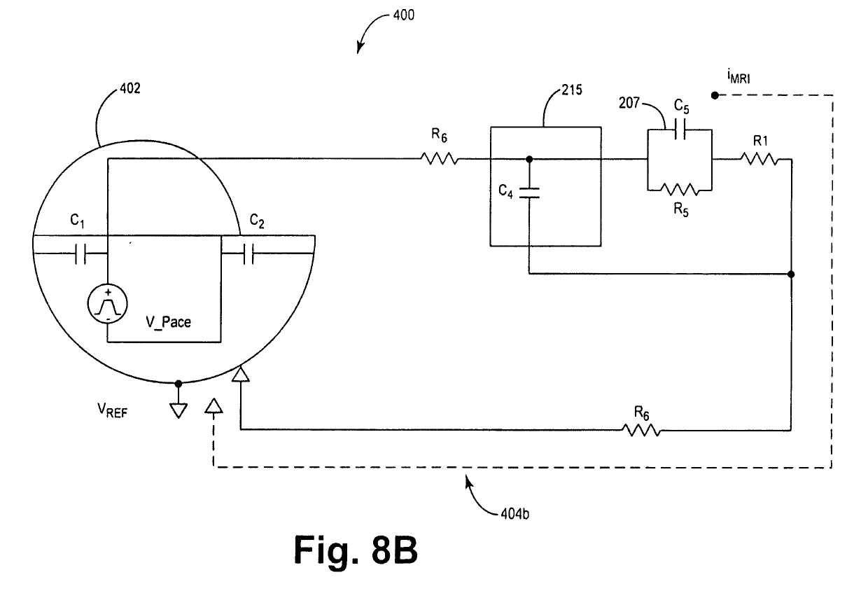

FIGS. 8A-8B depicts another simplified circuit 400 for a medical device system 100 during pacing and MRI conditions, respectively. Referring to FIG. 8A, circuit 400 includes an IMD circuit 402 (e.g. a pacemaker circuit, neurostimulator circuit etc.) and a bipolar shunted lead 304b. Circuit 400 includes the same elements as circuit 300 depicted in FIG. 7A, except magnetostrictive element 515 is coupled to a capacitor (C4) instead of inductor L. Specifically, C4 serves to bypass first and second elements 240a, 240b when a patient undergoes a MRI procedure. Magnetostrictive material 515 only acts as a switch to turn on and off C4 in circuit 400. In this embodiment, high frequency signals (i.e. from the MRI) pass to C4 whereas low frequency signals pass to and from tip electrode 207. C4 is shorted when exposed to high frequency signals. C4 acts as an “open circuit” when exposed to low frequency signals, which causes the pacing pulses to pass directly to tip electrode 207. An exemplary value for C4 is about 1-10 uF. For the bipolar shunted lead circuit 404a depicted in FIG. 8A, i_{MRI} is transferred from tip electrode 207 through ring electrode 216 back to medical device housing 102, which avoids or substantially prevents the MRI from affecting the operation of lead 106 from delivering electrical stimuli (i.e. pacing pulses) to the patient.

Referring to FIG. 8B, unipolar circuit 404b is the same as bipolar circuit 404a except unipolar circuit 404b lacks ring electrode 216. When lead 106 is exposed to MRI, I_{MRI} is induced, as depicted by the ghost lines. As shown, the I_{MRI} is transferred from tip electrode 207 back to medical device housing 102. If the patient requires pacing during an MRI procedure, a pacing current is generated from C1 or C2 and passes through magnetostrictive element 215, tip electrode 207 back to medical device housing 102.

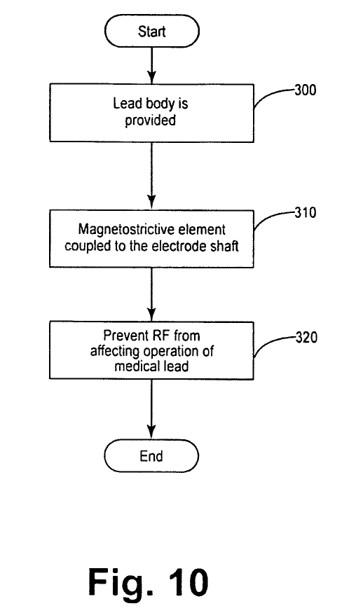

FIG. 10 is a flow diagram that depicts a method of producing a medical lead. At block 300, a lead body is provided. At block 310, a magnetostrictive element is inserted or placed between a lead body and an electrode shaft. The magnetostrictive element is comprised of a ferromagnetic material (e.g. terfenol-D, galfenol etc.), commercially available from Extrema Products Inc. located in Ames, Iowa. Terfenol-D, an alloy of terbium, dysprosium, and iron metals with the formula of terbium (Tb)(0.3) dysprosium (Dy)(0.7) iron (Fe)(1.9), was developed at the Naval Ordnance Laboratory in America. Generally, terfenol has the largest room temperature magnetostriction of any material. In mechanical terms, a 2.5 inch diameter rod of terfenol-D is capable of generating over 50,000 pounds of dynamic force. In one embodiment, the magnetostrictive element can comprise one or more terfenol-D or galfenol. In another embodiment, the magnetostrictive element can comprise terfenol-D and/or galfenol in combination with other suitable materials.

At block 320, the RF is prevented from affecting the sensing operation of the medical lead. In one embodiment, the magnetostrictive element reduces by at least 80 percent the current, induced in the lead by the MRI. In another embodiment, the magnetostrictive element reduces by at least 50 percent the current induced by the MRI.

It is understood that the present invention is not limited for use in pacemakers, cardioverters of defibrillators. Other uses of the leads described herein may include uses in patient monitoring devices, or devices that integrate monitoring and stimulation features. In those cases, the leads may include sensors disposed on distal ends of the respective lead for sensing patient conditions.

The leads described herein may be used with a neurological device such as a deep-brain stimulation device or a spinal cord stimulation device. In those cases, the leads may be stereotactically probed into the brain to position electrodes for deep brain stimulation, or into the spine for spinal stimulation. In other applications, the leads described herein may provide muscular stimulation therapy, gastric system stimulation, nerve stimulation, lower colon stimulation, drug or beneficial agent dispensing, recording or monitoring, gene therapy, or the like. In short, the leads described herein may find useful applications in a wide variety medical devices that implement leads and circuitry coupled to the leads.

Various embodiments of the invention have been described. These and other embodiments are within the scope of the following claims. For example, electrode 207 may include variously shaped electrodes such as ring shaped or other suitable shapes. Additionally, skilled artisans appreciate that other dimensions may be used for the mechanical and electrical elements described herein.

1. A medical device lead comprising:
a lead body;
an electrode shaft disposed inside of the lead body; and
a magnetostrictive element coupled to the electrode shaft.

2. The medical device lead of claim 1, wherein the magnetostrictive element comprises at least one of terfenol-D and galfenol.

3. The medical device lead of claim 1, wherein the magnetostrictive element being coupled to a first segment and a second segment of the electrode shaft.

4. The medical device lead of claim 3, wherein the magnetostrictive element causes at least one of the first segment and the second segment of the electrode shaft to move in response to magnetic resonance imaging (MRI).

5. The medical device lead of claim 4, wherein the second segment moves in the distal direction away from the first segment of the electrode shaft.

6. The medical device lead of claim 4, wherein current, induced in the lead by the MRI, passes to the magnetostrictive element.

7. The medical device lead of claim 6, wherein current, induced in the lead by the MRI, passes to a radiofrequency (RF) trap.

8. The medical device lead of claim 6, wherein current, induced in the lead by the MRI, does not pass to an electrode tip.

9. The medical lead of claim 8, wherein the current, induced in the lead by the MRI, is significantly reduced by use of a high frequency impedance component.

10. The medical lead of claim 9, wherein the current, induced in the lead by the MRI being significantly reduced by at least 80 percent.

11. The medical device lead of claim 1, further comprising a radiofrequency shunted sleeve head.

12. The medical device lead of claim 1, wherein the magnetostrictive element serves as a switch.

13. A medical device lead comprising:
a lead body;
a tip electrode coupled to the lead body,
a magnetostrictive element coupled to the lead body and to an electrode shaft, wherein the magnetostrictive element comprises at least one of terfenol-D and galfenol.

14. The medical device lead of claim 13, wherein the magnetostrictive element expands when exposed to MRI.

15. The medical device lead of claim 13, wherein the magnetostrictive element contracts when the magnetostrictive element is not exposed to MRI.

16. A medical device lead comprising:
a lead body;
an electrode shaft disposed inside of the lead body;
at least one conductive ring coupled to the electrode shaft;
a tip electrode coupled to the electrode shaft;
a magnetostrictive element coupled to the conductive member.

17. The medical device lead of claim 16, wherein the magnetostrictive element comprises at least one of terfenol-D and galfenol.

18. The medical device lead of claim 16, wherein the magnetostrictive element expands when exposed to MRI.

19. The medical device lead of claim 16, wherein the magnetostrictive element contracts when MRI is absent.

20. A medical device lead comprising:
a lead body;
an electrode shaft disposed inside of the lead body;
a magnetostrictive element coupled to the electrode shaft; and
one of an inductor and a capacitor coupled to the magnetostrictive element,
wherein the magnetostrictive element serves as a switch.

21. A bipolar shunted lead circuit associated with an implantable medical device comprising:
a first capacitor;
a first resistor in parallel with the first capacitor, the first resistor and the first capacitor associated with a tip electrode;
a second capacitor;
a second resistor in parallel with the second capacitor, the second resistor and the second capacitor associated with a ring electrode;
a magnetostrictive element coupled to the first resistor and the first capacitor;
the magnetostrictive element coupled to the second resistor and the second capacitor; and
a third capacitor associated with a magnetostrictive element.

22. The bipolar shunted lead circuit of claim 21, wherein the magnetostrictive element shunts current induced in the lead.

23. The bipolar shunted lead circuit of claim 22, wherein the magnetostrictive element being coupled to the third capacitor.

24. A unipolar shunted lead circuit associated with an implantable medical device comprising:
a first capacitor;
a first resistor in parallel with the first capacitor, the first resistor and the first capacitor associated with a tip electrode; and
a magnetostrictive element coupled to the first resistor and the first capacitor.

25. The unipolar shunted lead circuit of claim 24, further comprising:
another capacitor associated with the magnetostrictive element.
